(19) United States
(12) Patent Application Publication
(10) Pub. No.: US 2023/0103485 A1
(43) Pub. Date: Apr. 6, 2023

(54) IMPLANT SYSTEM

(71) Applicant: Karl Leibinger Medizintechnik GmbH & Co. KG, Muehlheim / Donau (DE)

(72) Inventors: Adem Aksu, Schwenningen (DE); Veit Irion, Mühlheim a. d. Donau (DE); Frank Reinauer, Emmingen-Liptingen (DE)

(21) Appl. No.: 17/936,905

(22) Filed: Sep. 30, 2022

(30) Foreign Application Priority Data
Oct. 4, 2021 (DE) 102021211157.2

(51) Int. Cl. A61F 2/30

(52) U.S. Cl. CPC A61F 2/3094; A61L 2430/02

(57) ABSTRACT

An implant system for treating fractures of a curved bone structure is proposed which comprises the following: an implant body which is provided to be arranged on the bone structure and to be in contact with the latter at least in some regions, wherein the implant body has a rod-like longitudinal extent, wherein the implant body has at least one middle piece which, at both ends, has at least one respective holding piece, wherein the holding pieces are each designed to be flexurally and/or torsionally resistant in such a way as to allow the respective holding piece to rest substantially flat along a particular region of the bone structure assigned to it, and wherein the at least one middle piece is designed to be elastic in such a way that the at least two holding pieces are bendable relative to each other about at least one flexion or torsion axis.

### PRIORITY CLAIM

This application claims priority to German application No. 102021211157.2, filed on Oct. 4, 2021, the entire disclosure of which is incorporated herein by reference.

### BACKGROUND

The present invention relates to an implant system for treating fractures of a curved bone structure, in particular broken ribs in the chest region. The invention further relates to a method for production of such an implant system.

US 2021/0298808 A1 discloses a sternum replacement plate having a central region and rod-like extensions. The extensions are thicker than the central region, such that the central region is bendable about a flexion axis.

US 2018/0360506 A1 discloses a bone fixation device having two rigid endpieces which are connected via an elastic connection region of wires.

A chest injury is understood as damage to the osseous thoracic cage and to all of the organs and organ systems contained in the latter. Chest injuries occur mainly in the context of violent impacts involving damage to multiple regions of the body. While isolated chest injuries are a rare occurrence, almost half of all patients suffering polytrauma present at the same time with a chest injury. Polytrauma is the term used when multiple regions of the body or multiple organ systems are affected, where one of the injuries on its own or the combination of the injuries is life-threatening. The chest is composed of the thoracic spine, the sternum, and twelve pairs of ribs. In the cranial direction, the chest is delimited by the superior aperture of the thoracic cavity, which is formed by the first thoracic vertebra, the two first ribs, and the upper part of the sternum. It constitutes the connection between the connective tissue spaces of the neck and of the thoracic cage. The caudal limit of the chest, the inferior aperture of the thoracic cavity, constitutes the boundary between the thoracic space and the abdominal space. It is formed by the 12th thoracic vertebra, the 11th and 12th ribs, the cartilage parts of the 7th to 10th ribs, and by the inferior margin of the sternum. The boundary structure between thoracic space and abdominal space is the diaphragm, which stretches between the aforementioned parts of the skeleton. The ribs are connected to the spinal column via the costovertebral joints and also to the breastbone via the sternocostal joints.

In interaction with the thoracic cage muscles, which lie between the ribs and fill the regions between the ribs, called the intercostal spaces, the joints play a central role in the mechanism of respiration. Since the thoracic cage also accommodates vital organs such as the heart and lungs and important pathways, it also serves as a protective framework.

During inhalation, expansion of the heads of the ribs in the costovertebral joints causes the ventral sternal ends of the ribs to lift. In parallel with this, the diaphragm lies deeper as a result of contraction. This results in an increased volume of the chest. During exhalation, the ventral sternal ends of the ribs fall again and the diaphragm muscles relax, such that the chest returns to its original volume. Of all the fractures in the chest region, rib fractures are the most common.

Fractures of this kind, such as those of the ribs, can be fitted for example with metal implants for treatment and bone reconstruction. Use is made, for example, of Kirschner wires which, in a procedure called K-wire osteosynthesis, are able to fix the fractures in question. However, after being introduced into the bone, these wires can sometimes migrate, particularly into bone cavities, and they are also not free from failure. Moreover, when using K-wires, immobilization often requires additional fixing by plaster cast, which makes the treatment of chest injuries more difficult.

Fractures can also be treated using metal splints, but these permit only insufficient suppression of pain. In the case of rib fractures, this suppression of pain cannot even be fully achieved by using rib clips, and these moreover do not provide for a controlled movement in the fracture gap. Overall, primary bone healing is at any rate also very difficult here.

A further possible way of fixing the aforementioned fractures is by plate osteosynthesis by means of plates or splints. In this context, DE 38 03 435 C1 discloses an implant splint with a side face that is adapted to the contour of a rib. A possible disadvantage of plate osteosynthesis is that the respiratory mechanism of the thorax may be impaired by the stiffness of the implant. Particularly in the case of bones with osteoporosis, loosening of screw-type fastenings may also occur at the transition to the non-rigid rib region.

### SUMMARY OF THE DISCLOSURE

The object of the present invention is therefore to make available an implant system which treats a fracture of a bone structure safely and in a manner free from failure and, while providing good bone healing and suppression of pain, at the same time permits a controlled movement in the fracture gap.

With the implant system according to the invention, an implant body can be arranged on the curved bone structure in a way which, for example by bending, imitates the curvature of the bone structure or at least partially encloses or surrounds the bone structure by twisting of the at least one middle piece, such that, on the one hand, the fragments of the bone structure are securely held and, on the other hand, a controlled movement of the bone structure as a whole is permitted without impeding the healing process, which is to say that the implant system permits a controlled elastic movement of the affected bone fragments of the bone structure. By virtue of the elastic configuration of the at least one middle piece, the holding pieces can be arranged in different positions relative to each other on the bone structure and optionally secured, whereas the middle piece or middle pieces is/are flexibly molded onto the bone structure. The implant system according to the invention is particularly suitable for treating damaged bone structures in the chest region, i.e. damaged rib bones, but is not limited thereto.

An embodiment with a middle piece and with two holding pieces located at the ends of the latter has already been described, but other configurations are also conceivable. Thus, the holding pieces can be adjoined by further elastic middle pieces, which in turn can be followed again by holding pieces. It is also conceivable for one or other piece to be omitted, such that a juxtaposition of middle pieces and holding pieces is conceivable which can engage once or even several times around a wide variety of bone structures of changing circumference and of great longitudinal extent.

In an advantageous development of the implant system according to the invention, which has a high degree of flexibility and permits good handling in terms of the arrangement of the implant body on the bone structure, the implant body, with its rod-like longitudinal extent, can have at least one region whose side edges have an undulating configuration, in particular a uniformly undulating configuration, on at least two mutually opposite sides. In this case, the at least one middle piece and also one or more holding pieces can be designed individually, but also jointly, with the aforementioned undulating shape.

Particularly preferably, in a development permitting simplified production, the at least one middle piece and the at least two holding pieces can have substantially the same outer contour, that is to say, for example, the identical uniform undulating shape at their side edges. However, in terms of their shape, the holding pieces can also differ from one another and also in each case from the at least one middle piece.

In one advantageous embodiment, a possibility of adapting the properties of the implant body of the implant system according to the invention to the conditions of the bone structure, and to the degree of damage of the latter, is one in which the thickness and/or length of the holding pieces and/or of the middle piece are different and/or variable. The variability of the holding pieces and middle piece means that the implant body in question is able to be individually adapted by measuring the respective situation. In simplified developments of implant bodies, the holding pieces for example can be designed identically in pairs.

In one development, in which on the one hand the middle piece affords a high degree of flexibility and, on the other hand, the holding pieces are very stable, the holding pieces can be designed with an increased thickness in relation to the at least one middle piece, such that they have a reduced pliability.

In order, on the one hand, to be able to connect the middle piece and the holding pieces of the implant body, particularly when the pieces have different thicknesses, in a way that does not require any unnecessary projections or steps, and, on the other hand, if necessary or desired, to provide the holding pieces with a preferred orientation by arranging them at an angle to the at least one middle piece, an advantageous development of the implant system according to the invention is one in which a transition region can be provided between at least one of the holding pieces and the at least one middle piece. For this purpose, the transition region can, for example, have at least one portion arranged at an angle to the longitudinal extent, although the transition region can also form a simple narrowing of the thickness of the cross section from the thickness of the at least one middle piece to the thickness of the respective holding piece. A transition region is likewise conceivable in which a flat face of the at least one middle piece and a flat face of the holding pieces, in particular the flat face for resting on the bone structure, lie in one plane.

As regards their dimensions, the middle piece and the holding pieces are intended to be subject to no limitations other than those of the environment in which they are used. However, with respect to individual directions of extent, certain dimensions are particularly preferred. Thus, the length of the implant body can preferably be between 1 mm and 300 mm, the length of the at least one middle piece can be between 1 mm and 50 mm, its thickness can be between 0.1 mm and 3.5 mm, while the thickness of the holding pieces can be 0.5 mm to 4.5 mm. The corners formed in the case of an undulating configuration of the side edges of the implant body can have rounded shapes in the region of between 0.01 and 4.5 mm. Moreover, it is conceivable that the stated dimensions can vary over their respective directions of extent.

In order to easily hold the implant body firmly in place in an expedient development of the implant system according to the invention, at least one fastening means can be arranged between the implant body and the bone structure.

The at least one fastening means is preferably formed with one or several spikes, points, hooks or similar projections which are configured to penetrate into the bone structure. Projections of this kind can be provided both on the at least one middle piece and on the two holding pieces, and both on the face directed toward the bone structure, where they penetrate into the bone structure, and on the face directed away from the bone structure, where they are able to fix the surrounding tissue. Another preferred development may be one in which screws engage, by way of receptacles provided for this purpose, on one or more of the holding pieces and thus fasten these to the bone structure. Receptacles for the passage of screws or of similar fastening means can also be provided on middle pieces.

In order for the particular and if appropriate individually manufactured implant body to be able to be suitably adapted to the particular situation, for example in terms of specifically chosen material properties such as fatigue resistance, flexibility, ideal elastic modulus or strength, another advantageous development of the implant system according to the invention is one in which the implant body can be made from at least one high-performance polymer or composites thereof. Particularly preferably, the at least one middle piece and the holding pieces can each be made from a thermoplastic such as a polyether ether ketone (PEEK) material, a polyether ketone ketone (PEKK) material, a polyphenyl sulfone (PPSU) material, a polyethylene (PE) material and compounds of the abovementioned materials with hydroxyapatite, tricalcium phosphate, strontium, magnesium and the like. However, further materials are also conceivable, and the above list is not exhaustive.

Particularly in the case of individual manufacture of the implant body, for example for treating complicated fractures or those with a poor healing prognosis, the at least one middle piece and the holding pieces can have, on their face in each case directed toward the bone structure, a surface that is adapted to the associated bone region and that permits still more exact adaptation.

Moreover, the object can also be achieved by a method for production of an implant system for treating fractures of a curved bone structure. It is likewise conceivable to produce the final shape of the implant body, and also the final shape of the at least one elastic middle piece, by reworking an implant body blank that has been obtained in a primary forming method. Such reworking can, for example, involve a cutting operation in which the blades of a tool are used to mechanically cut away material from the blank in order to change the workpiece shape and (or) workpiece surface. The material removal can take place for example as a milling procedure using geometrically defined blades; such a manufacturing method can permit a high degree of manufacturing precision.

In this way too, an implant system can be made available which treats a fracture of a bone structure safely and in a manner free from failure and, while providing good bone healing and suppression of pain, at the same time permits a controlled movement in the fracture gap.

The embodiments and developments above may be combined arbitrarily with one another insofar as is rational. Further possible embodiments, developments and implementations of the invention also encompass combinations, not explicitly stated, of features of the invention that are described above or below in relation to the working examples.

### DESCRIPTION OF THE DRAWINGS The invention is explained in more detail below on the basis of exemplary embodiments shown schematically in the figures of the drawing, in which:

FIG. 1a shows a perspective side view of a first embodiment of the implant system according to the invention with an implant body in extended form;

FIG. 1b shows a perspective side view of the implant body from FIG. 1a in a form in which it is bent about a flexion axis;

FIG. 1c shows a perspective side view of the implant body from FIG. 1a when arranged on the bone structure of a costal arch;

FIG. 1d shows a perspective side view of the implant body from FIG. 1c when seen from below;

FIG. 2a shows a perspective side view of a second embodiment of the implant system according to the invention with an implant body in extended form, in which, on the flat face directed away from the bone structure, the holding pieces are provided with points, but the middle piece has no points;

FIG. 2b shows a perspective side view of a detail region of the one holding piece of the implant body from FIG. 2a;

FIG. 2c shows a perspective side view of the implant body from FIGS. 2a and 2b, in which, on the flat face of the implant body directed away from the bone structure, both the holding pieces and the middle piece are provided with points;

FIG. 3a shows a perspective side view of a third embodiment of the implant system according to the invention with an implant body in extended form, in which, on the flat face directed away from the bone structure, the middle piece is provided with points;

FIG. 3b shows a perspective side view of a detail region of the middle piece of the implant body from FIG. 3a;

FIG. 3c shows a perspective side view of the implant body from FIGS. 3a and 3b, in which, on the flat face of the implant body directed toward the bone structure, only the middle piece is provided with points, and the faces of the middle piece and of the holding pieces lie in one plane;

FIG. 3d shows a perspective side view of a detail region of the middle piece of the implant body from FIGS. 3a-3c;

FIG. 4 shows a perspective side view of a fourth embodiment of the implant system according to the invention with an implant body in extended form, in which the holding pieces are configured with an undulating shape at their side edges, whereas the middle piece extends with parallel straight side edges; and

FIG. 5 shows a perspective side view of a fifth embodiment of the implant system according to the invention with an implant body in extended form, in which the holding pieces, at their side edges, are configured substantially with straight parallel side edges, whereas the middle piece extends straight but with undulating side edges.

In all of the figures, identical or functionally identical elements and devices are provided with the same reference signs, unless indicated otherwise.

### DETAILED DESCRIPTION

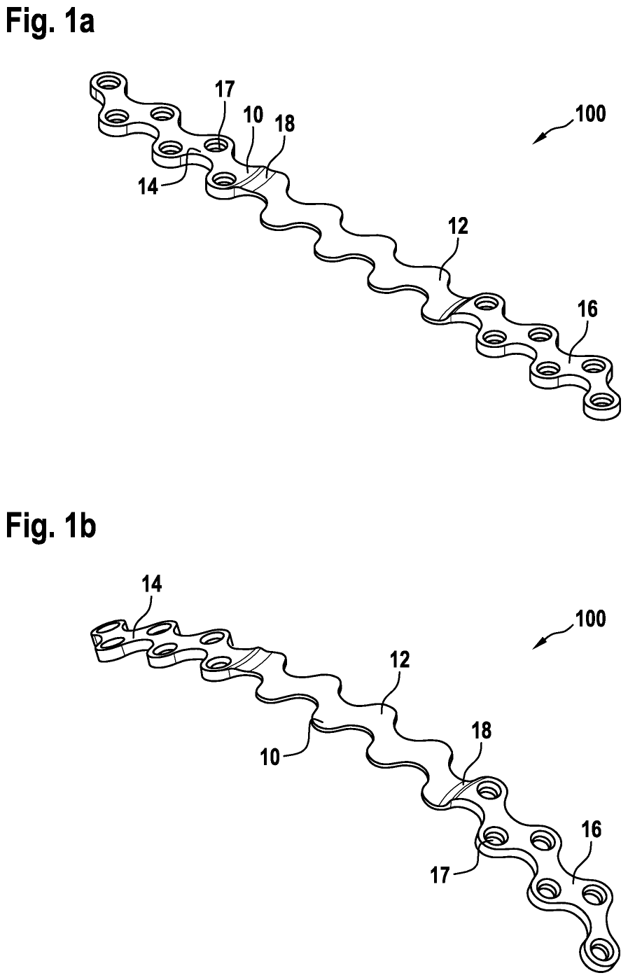

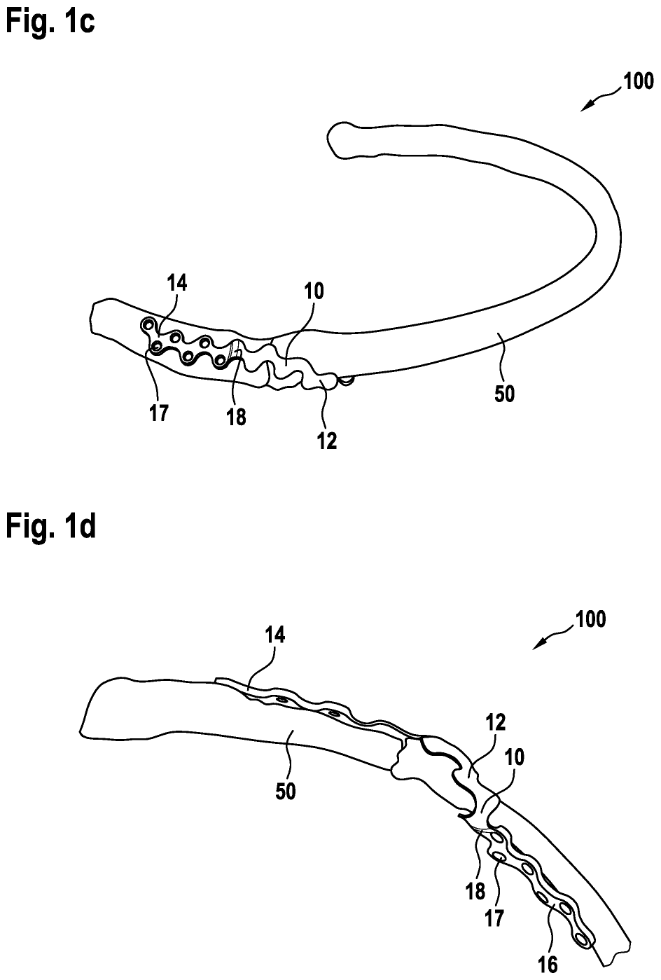

FIGS. 1 to 5 of the drawings show an implant system 100 for treating fractures of a curved bone structure 50, which system comprises the following: an implant body 10, which is provided to be arranged on the bone structure 50 and to be in contact with the latter at least in some regions, wherein the implant body 10 has a rod-like longitudinal extent, wherein the implant body 10 has at least one middle piece 12 which, at both ends, has at least one holding piece 14, 16, wherein the holding pieces 14, 16 are each designed to be flexurally and/or torsionally resistant in such a way as to allow the respective holding piece 14, 16 to rest substantially flat along a particular region of the bone structure 50 assigned to it, and wherein the at least one middle piece 12 is designed to be elastic in such a way that the at least two holding pieces 14, 16 are bendable relative to each other about at least one flexion or torsion axis.

FIG. 1a shows a perspective side view of a first embodiment of the implant system 100 according to the invention with an implant body 10 in extended form. The implant body 10 is configured with a rod-like longitudinal extent and, both at the middle piece 12 and at the two holding pieces 14, 16 having the same outer contour, is provided with side edges which have a uniformly undulating shape at two mutually opposite sides. The undulating shape at the side edges results in the formation of an alternating series of projecting and recessed portions that merge into one another, wherein a recessed portion of one side edge is coincident with a projecting portion of the opposite side edge. In the projecting portions of the holding pieces 14, 16, receptacles 17 for the arrangement of fastening screws (not shown) are introduced perpendicularly with respect to the longitudinal extent. Arranged between the holding pieces 14, 16 and the middle piece 12 there is in each case a transition region 18, where a stepless transition is produced between the thicker holding pieces 14, 16, i.e. thicker perpendicular to the longitudinal extent, and the thinner middle piece 12. The transition pieces transfer the levels of the flat faces of the holding pieces 14, 16, which are flush with one another when the implant body 10 is not bent, into the level of the middle piece 12.

FIG. 1b shows a perspective side view of the implant body 10 from FIG. 1a in a shape in which it is bent about a flexion axis, wherein the flexion axis (not shown) in the plane defined by the middle piece 12 runs centrally and transversely to the longitudinal extent of the implant body 10.

FIG. 1c shows a perspective side view of the implant body 10 from FIG. 1a arranged on the damaged bone structure 50 of a costal arch. The damage to the bone structure 50, in the form of a fracture, can be clearly seen. The figure also shows one of the holding pieces 14, which is fastened to the bone structure 50, and also the middle piece 12 which is twisted about a torsion axis extending along the bone structure, in such a way that the implant body 10 wraps around the bone structure 50 through approximately 180°. FIG. 1d shows a perspective side view of the implant body from FIG. 1c in a view from below, in which both holding pieces 14, 16 and the middle piece 12 can be seen. FIGS. 1c and 1d also show that the holding pieces 14, 16 are spaced apart from the fracture itself, and the middle piece 12 wraps or engages around this region in such a way that, while promoting healing of the bone, it promotes a controlled movement and load distribution of the dynamic chest cavity.

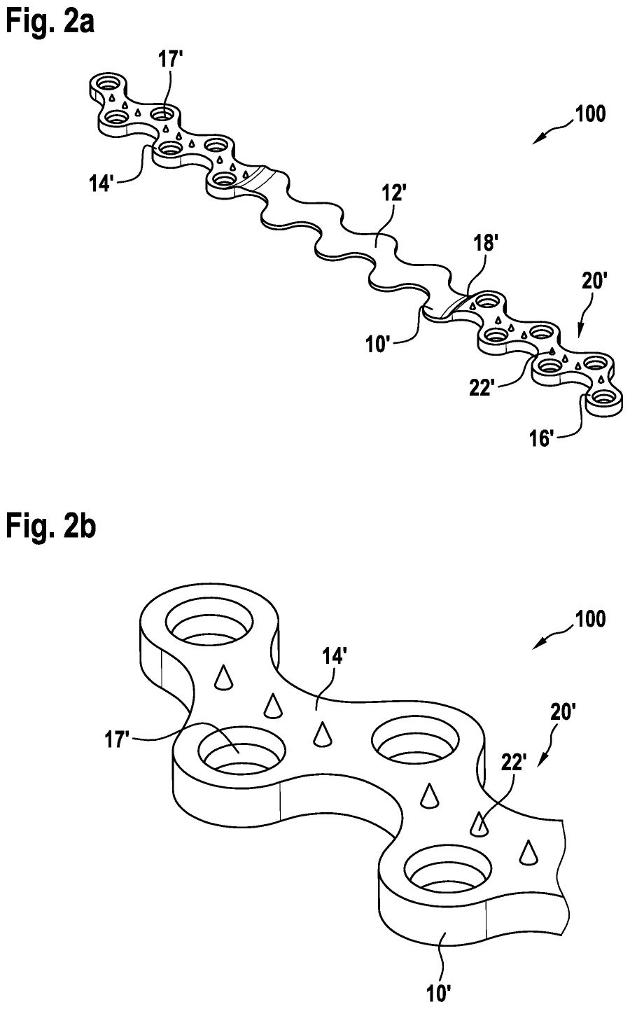

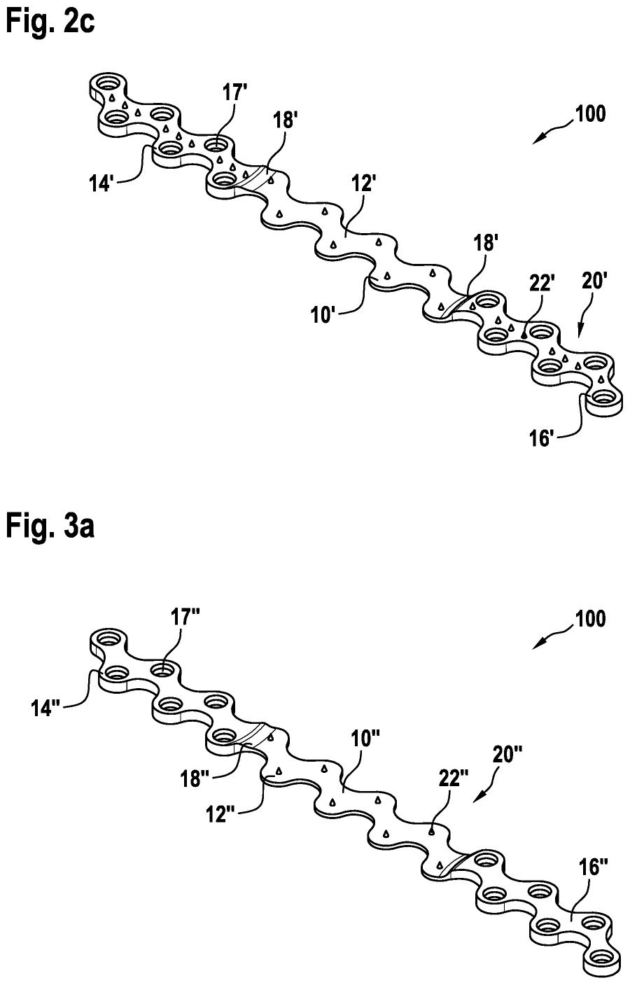

FIG. 2a shows a perspective side view of a second embodiment of the implant system 100 according to the invention with an implant body 10′ in extended form, in which, on the flat face directed away from the bone structure 50 (not shown), the holding pieces 14′, 16′ are provided with points 22′ as fastening means 20′, whereas the middle piece 12 has no points 22′ on its flat face. In the implant body 10′, which again has a rod-like longitudinal extent, the middle piece 12′ and the holding pieces 14′, 16′ are once more provided with side edges that have a uniformly undulating shape at two mutually opposite sides. Here too, between the holding pieces 14′, 16′ and the middle piece 12′, there is in each case a transition region 18′, where a stepless transition is produced between the thicker holding pieces 14′, 16′ and the thinner middle piece 12′.

FIG. 2b shows a perspective side view of a detail region of the one holding piece 14′ of the implant body 10′ from FIG. 2a, showing that the points 22′ are arranged, in the direction of extent of the implant body 10′, in respective groups of three points 22′ along a line between the projecting and recessed portions. Here too, receptacles 17′ for the arrangement of fastening screws (not shown) are introduced into the projecting portions of the holding pieces 14′, 16′ perpendicularly with respect to the longitudinal extent.

FIG. 2c shows a perspective side view of the implant body 10′ from FIGS. 2a and 2b, in which, on the flat face of the implant body 10′ directed away from the bone structure, both the holding pieces 14′, 16′ and the middle piece 12′ are provided with points 22′, as a result of which the tissue surrounding the implant body 10′ is held in place.

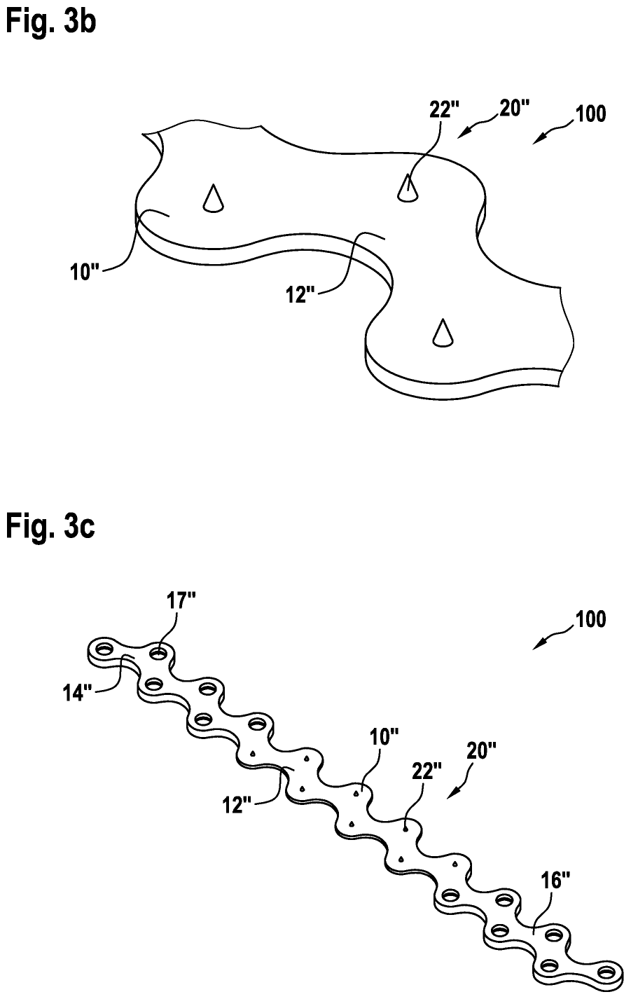

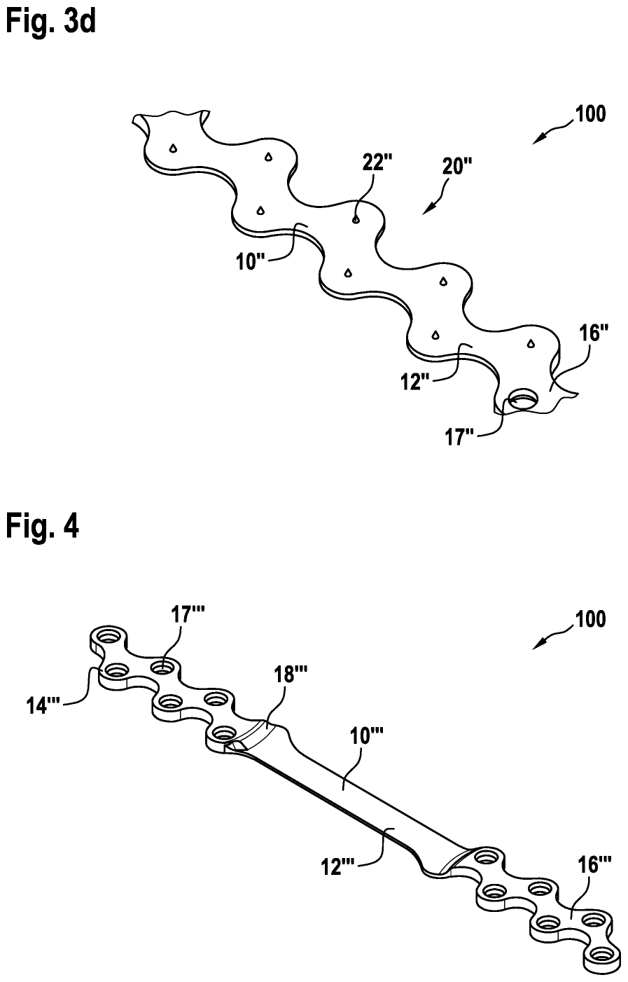

FIG. 3a shows a perspective side view of a third embodiment of the implant system 100 according to the invention with an implant body 10″ in extended form, in which, on the flat face directed away from the bone structure, the middle piece 12″ is provided with points 22′. Once again, the implant body 10″ is configured with a rod-like longitudinal extent and, both on the middle piece 12″ and on the two holding pieces 14″, 16″ having the same outer contour, is provided with side edges which have a uniformly undulating shape at two mutually opposite sides. The undulating shape at the side edges results in the formation of an alternating series of projecting and recessed portions that merge into one another, wherein a recessed portion of one side edge is coincident with a projecting portion of the opposite side edge. In the projecting portions of the holding pieces 14″, 16″, receptacles 17″ for the arrangement of fastening screws (not shown) are introduced perpendicularly with respect to the longitudinal extent.

FIG. 3b shows a perspective side view of a detail region of the middle piece 12″ of the implant body 10″ from FIG. 3a. It shows that the points 22″ on the middle piece 12″ are in each case arranged individually on each projecting portion.

FIG. 3c shows a perspective side view of the implant body 10″ from FIGS. 3a and 3b, in which, also on the flat face of the implant body 10″ directed toward the bone structure, only the middle piece 12″ is provided with points 22′, and the faces of the middle piece 12″ and of the holding pieces 14″, 16″ lie in one plane.

FIG. 3d shows a perspective side view of a detail region of the middle piece 12″ of the implant body 10″ from FIGS. 3a-3c.

FIG. 4 shows a perspective side view of a fourth embodiment of the implant system 100 according to the invention with an implant body 10′″ in extended form, in which the holding pieces 14′″, 16′″ are configured with an undulating shape at their side edges, whereas the middle piece 12′″ extends with parallel straight side edges. Here too, a transition region 18′″ is arranged between each of the holding pieces 14′″, 16′″ and the middle piece 12′″. Regarding the undulating shape of the holding pieces 14′″, 16′″ and the receptacles 17′″ for fastening means, reference may be made to what has been said concerning FIGS. 1 to 3.

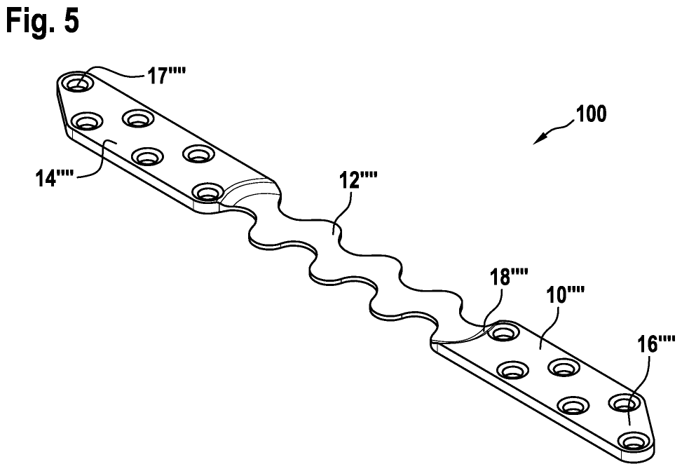

FIG. 5 shows a perspective side view of a fifth embodiment of the implant system 100 according to the invention with an implant body 10″″ in extended form, in which the holding pieces 14″, 16″″, at their side edges, are configured substantially with straight parallel side edges, whereas the middle piece 12′ extends straight but with undulating side edges. The receptacles 17′ for the arrangement of fastening screws have the same pattern here as in the case of an undulating configuration of the side edge and, at the ends of the holding pieces 14″, 16″″, their side edges converge at a receptacle 17″″.

The implant system 100 according to the invention solves the problem mentioned at the outset through the combination of an innovative and flexible design and the specifically chosen material properties, such as fatigue resistance, flexibility, ideal elastic modulus, material strength. It is thus possible to ensure a controlled load distribution and also controlled movement of the dynamic chest cavity, which, in contrast to a stiffer metallic system, promotes bone healing.

Although the present invention has been described above on the basis of preferred exemplary embodiments, it is not limited thereto and instead can be modified in a variety of ways. In particular, the present invention can be changed or modified in a variety of ways without departing from the core of the invention.

1. An implant system for treating fractures of a curved bone structure, comprising:
an implant body configured to be arranged on the bone structure and to be in contact with the bone structure at least in some regions, wherein the implant body has a rod-like longitudinal extent,
wherein the implant body has a middle piece which, at both ends, has a respective holding piece,
wherein each holding piece pieces is designed to be flexurally and/or torsionally resistant in such a way as to allow the respective holding piece to rest substantially flat along a particular region of the bone structure assigned to it, and
wherein the middle piece is designed to be elastic in such a way that the at least two holding pieces are bendable relative to each other about a flexion axis and a torsion axis, with the result that the middle piece is able to wrap around the bone structure at least in some regions.

2. The implant system as claimed in claim 1, wherein the implant body, with its rod-like longitudinal extent, has at least one region whose side edges have an undulating configuration on at least two mutually opposite sides.

3. The implant system as claimed in claim 1, wherein the middle piece and the two holding pieces have substantially the same outer contour.

4. The implant system as claimed in claim 1, wherein the thickness and/or length of the holding pieces and/or of the middle piece differ.

5. The implant system as claimed in claim 1, wherein the holding pieces have an increased thickness in relation to the at least one middle piece.

6. The implant system as claimed in claim 1, wherein a transition region is provided between at least one of the holding pieces and the middle piece.

7. The implant system as claimed in claim 6, wherein the transition region has at least one portion at an angle with respect to the longitudinal extent.

8. The implant system as claimed in claim 1, wherein at least one fastening means is arranged on the implant body.

9. The implant system as claimed in claim 8, wherein the fastening means is formed with one or more spikes, points, hooks or projections which are configured to penetrate into an osseous structure of the bone structure or to hold in place tissue that surrounds the implant body.

10. The implant system as claimed in claim 1, wherein the implant body is made from at least one high-performance polymer or composite thereof.

11. The implant system as claimed in claim 10, wherein the at least one high-performance polymer is a thermoplastic.

12. The implant system as claimed in claim 1, wherein the middle piece and the holding pieces have, on their face in each case directed toward the rib bone, a surface that is adapted to the associated bone region.

13. A method for production of an implant system for treating fractures of a curved bone structure, comprising at least the following production steps:
making available an implant body mold which is divided into at least three different regions, in particular a middle region, and an end region located at each of the ends of the middle region;
injecting at least one high-performance polymer into each of the regions of the implant body mold;
cooling and/or crosslinking the high-performance polymers in the different regions in order to connect the regions to give the rod-shaped, flexible implant body with the elastic middle piece and the two holding pieces which are bendable relative to each other about the one flexion axis and the one torsion axis;
removing the implant body from the implant body mold.

14. A method for production of an implant system as claimed in claim 1, wherein the flexible implant body with the elastic middle piece and with the two holding pieces, which are bendable relative to each other about the one flexion axis and the torsion axis, is produced in an additive method.

15. The implant system of claim 11, wherein the thermoplastic is selected from the group including a polyether ether ketone (PEEK) material, a polyether ketone ketone (PEKK) material, a polyphenyl sulfone (PPSU) material and a polyethylene (PE) material.

16. The implant system as claimed in claim 10, wherein the at least one high-performance polymer is a compound of a thermoplastic with one or more of hydroxyapatite, tricalcium phosphate, strontium, magnesium.

17. The implant system of claim 16, wherein the thermoplastic is selected from the group including a polyether ether ketone (PEEK) material, a polyether ketone ketone (PEKK) material, a polyphenyl sulfone (PPSU) material and a polyethylene (PE) material.
